(19) United States
(12) Patent Application Publication
(10) Pub. No.: US 2011/0166154 A1
(43) Pub. Date: Jul. 7, 2011

(54) IDENTIFICATION OF PREDICTIVE MARKERS OF RESPONSE TO DASATINIB IN HUMAN COLON CANCER

(75) Inventors: Dennis J. Slamon, Woodland Hills, CA (US); Richard S. Finn, Encino, CA (US); Judy Dering, Westlake Village, CA (US); J. Randolph Hecht, Los Angeles, CA (US); Zev A. Wainberg, Los Angeles, CA (US); Edwin A. Clark, Pennington, NJ (US); Charles L. Ginther, Los Angeles, CA (US)

(73) Assignee: BRISTOL-MYERS SQUIBB COMPANY, Princeton, NJ (US)

(21) Appl. No.: 12/863,207

(22) PCT Filed: Jan. 23, 2009
(86) PCT No.: PCT/US09/31860

§ 371 Date: Mar. 23, 2011

Related U.S. Application Data
(60) Provisional application No. 61/023,796, filed on Jan. 25, 2008.

(51) Int. Cl. A61K 31/506; C12Q 1/02; C12Q 1/68; A61P 35/00

(52) U.S. Cl. 514/252.19; 435/29; 435/6.11; 435/6.1

(57) ABSTRACT

Methods and compositions are provided useful in the diagnosis, treatment and management of cancers wherein cancer cells express certain polynucleotides and/or polypeptides that are differentially expressed in colon cancer subtypes that are sensitive to therapy with abl kinase inhibitors and src kinase inhibitors. In particular, PTK-7, PLK-2 and PLK-3 could be identified as markers for determining the responsiveness of cancer to dasatinib.

### REFERENCE TO RELATED APPLICATIONS

This application claims priority to U.S. Provisional application No. 61/023,796, filed Jan. 25, 2008, under 35 U.S.C. 119(e). The entire teachings of the referenced application are incorporated herein by reference.

### FIELD

The invention relates to methods and compositions useful in the diagnosis, treatment and management of cancers that express particular polynucleotides and/or polypeptides.

### BACKGROUND

Despite recent improvements, colorectal cancer remains the second most common cause of cancer death in the United States. Decades of study have identified the oncogene Src as playing an important role in colorectal malignancies. While mutations in the oncogene itself are uncommon, Src plays a role by modulating transduction from multiple inputs such as epidermal growth factor receptor (EGFR) family receptors, platelet-derived growth factor receptor (PDGFR), fibroblast growth factor receptors (FGFRs) as well as integrins, and cell-cell adhesion molecules (FIG. 1).

Dasatinib is an orally active, multi-targeted tyrosine kinase inhibitor of the ABL and Src family kinases and is currently being investigated in solid tumors (Table 1). Pre-clinical studies have identified potential biomarkers associated with response to dasatinib in breast cancer and other cancers in vitro (3,4,5). Dasatinib has been approved by the FDA for the treatment of chronic myelogenous leukemia (CML). Currently a need exists in the art to develop compounds that target specific molecular alterations in cancer cells to lead to better outcomes. However, critical to the development of these agents is the identification of patients most likely to respond to any given agent. To better understand the potential role of dasatinib in colon cancer, a panel of 27 human colon cancer cell lines which have undergone baseline microarray analysis were used.

### SUMMARY

The present invention relates to methods of using certain polynucleotides and/or polypeptides that are differentially expressed in colon cancer subtypes to predict sensitivity or resistance to therapy with abl kinase inhibitors and src kinase inhibitors, collectively protein tyrosine kinase inhibitors. In particular, the present inventors have discovered that PTK-7, PLK-2, and PLK-3 polynucleotides and/or polypeptides are differentially expressed in colon cancer subtypes that are sensitive to therapy with N-(2-chloro-6-methylphenyl)-2-(6-(4-(2-hydroxyethyl)piperazin-1-yl)-2-methylpyrimidin-4-ylamino)thiazole-5-carboxamide. In a specific embodiment of the present invention, elevated expression of one or more of PTK-7, PLK-2 and/or PLK-3 in a colon cancer cell or cell line, is indicative of said cell or cell line as having an increased likelihood of responding favorably to a protein tyrosine kinase inhibitor, such as dasatinib.

As is known in the art, N-(2-chloro-6-methylphenyl)-2-[[6-[4-(2-hydroxyethyl)-1-piperazinyl]-2-methyl-4-pyrimidinyl]amino]-5-thiazolecarboxamide refers to a compound having the following structure (I):

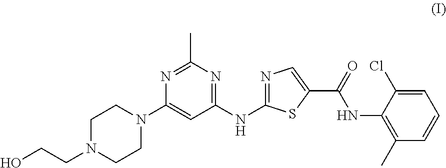

Compound (I) can also be referred to as N-(2-chloro-6-methylphenyl)-2-((6-(4-(2-hydroxyethyl)-1-piperazinyl)-2-methyl-4-pyrimidinyl)amino)-1,3-thiazole-5-carboxamide in accordance with IUPAC nomenclature. Use of the term encompasses (unless otherwise indicated) solvates (including hydrates) and polymorphic forms of the compound (I) or its salts (such as the monohydrate form of (I) described in U.S. Ser. No. 11/051,208, filed Feb. 4, 2005, incorporated herein by reference in its entirety and for all purposes. Pharmaceutical compositions of N-(2-chloro-6-methylphenyl)-2-[[6-[4-(2-hydroxyethyl)-1-piperazinyl]-2-methyl-4-pyrimidinyl]amino]-5-thiazolecarboxamide include all pharmaceutically acceptable compositions comprising N-(2-chloro-6-methylphenyl)-2-[[6-[4-(2-hydroxyethyl)-1-piperazinyl]-2-methyl-4-pyrimidinyl]amino]-5-thiazolecarboxamide and one or more diluents, vehicles and/or excipients, such as those compositions described in U.S. Ser. No. 11/402,502, filed Apr. 12, 2006, incorporated herein by reference in its entirety and for all purposes. One example of a pharmaceutical composition comprising N-(2-chloro-6-methylphenyl)-2-[[6-[4-(2-hydroxyethyl)-1-piperazinyl]-2-methyl-4-pyrimidinyl]amino]-5-thiazolecarboxamide is SPRYCEL™ (Bristol-Myers Squibb Company). SPRYCEL™ comprises N-(2-chloro-6-methylphenyl)-2-[[6-[4-(2-hydroxyethyl)-1-piperazinyl]-2-methyl-4-pyrimidinyl]amino]-5-thiazolecarboxamide, also referred to as dasatinib, as the active ingredient, and as inactive ingredients or excipients, lactose monohydrate, microcrystalline cellulose, croscarmellose sodium, hydroxypropyl cellulose, and magnesium stearate in a tablet comprising hypromellose, titanium dioxide, and polyethylene glycol.

Dasatinib (in some references also referred to as BMS-354825) is a highly potent, oral multi-targeted kinase inhibitor that targets BCR-ABL and SRC kinases with IC50s for the isolated kinases of 0.55 and 3.0 nM, respectively. Dasatinib is an orally active small molecule kinase inhibitor of both the src and abl proteins. It is a thiazole- and pyrimidine-based SFK/Abl kinase inhibitor (see, e.g., Nam, Sangkil et al., Cancer Research 65, 9185-9189, Oct. 15, 2005; Lombardo L J et al., J Med Chem, 2004 Dec. 30;47(27): 6658-61; and NDA 21-986, ODAC briefing document). Dasatinib also inhibits other oncogenic kinases such as c KIT, platelet-derived growth factor receptor, and ephrin A receptor kinases). In contrast to imatinib, which binds only to the inactive conformation, dasatinib binds to both the active or “opened” conformation and the inactive or “closed” conformation of the ABL kinase domain of BCR-ABL.

The structure and use of N-(2-chloro-6-methylphenyl)-2-[[6-[4-(2-hydroxyethyl)-1-piperazinyl]-2-methyl-4-pyrimidinyl]amino]-5-thiazolecarboxamide as an anticancer agent is described in Lombardo, L. J., et al., J. Med. Chem., 47:6658-6661 (2004) and is described in the following US patents and pending applications: U.S. Pat. No. 6,596,746, granted Jul. 22, 2003; U.S. Ser. No. 10/395,503, filed Mar. 24, 2003, all of which are incorporated by reference herein in their entirety.

Provided herein are methods for determining the responsiveness of a mammalian tumor cell to treatment with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof, which comprise determining the level of expression of at least one polynucleotide or polypeptide selected from PTK-7, PLK-2, and/or PLK-3 in said tumor cell, wherein the level of expression is indicative of whether the cell is likely to respond or is responsive to the treatment. In one aspect, the difference in the level of said one polynucleotide or polypeptide is a difference in the mRNA level (measured, for example, by RT-PCR or a microarray), such as at least about a two-fold difference, at least about a three-fold difference, or at least about a four-fold difference in the level of expression, or more. In another aspect, the difference in the level of the biomarker is determined at the protein level by mass spectral methods or by FISH or by IHC. In another aspect, the difference in the level of the biomarker refers to a p-value of <0.05 in Anova analysis. In yet another aspect, the difference is determined in an ELISA assay.

A kit for use in characterizing a mammalian tumor cell is also provided which comprises a means for determining the level of expression of at least one polynucleotides and/or polypeptides selected from PTK-7, PLK-2, and/or PLK-3 in said tumor cell, and instructions for use and interpretation of the kit results.

Additional embodiments relate to methods for determining the responsiveness of an individual with cancer to treatment with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof. These methods comprise obtaining a biological sample from said individual; and determining the level of expression of at least one polynucleotide and/or polypeptide selected from PTK-7, PLK-2, and/or PLK-3 in the biological sample, wherein said level of expression is indicative of responsiveness to the treatment.

Further provided are methods for determining the responsiveness of an individual with cancer to treatment with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof; comprising obtaining a biological sample from said individual; and determining the level of expression of at least one polynucleotide and/or polypeptide selected from PTK-7, PLK-2, and/or PLK-3 in the biological sample, wherein said level of expression is indicative of responsiveness to the treatment.

The application also provides methods of treating an individual suffering from a cancer which comprise determining the level of expression of at least one polynucleotide and/or polypeptide selected from PTK-7, PLK-2, and/or PLK-3 in a biological sample obtained from the subject; and administering N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof to the individual if the at least one polynucleotide and/or polypeptide is present in the biological sample at an increased level as compared to a control sample.

Additionally, the application provides methods of evaluating tumor aggressiveness which comprise determining the level of expression of at least one polynucleotide and/or polypeptide selected from PTK-7, PLK-2, and/or PLK-3 in a tumor sample and correlating the level of expression to tumor aggressiveness.

### BRIEF DESCRIPTION OF THE DRAWINGS

FIG. 1 shows regulation of transcription and translation of Src.

FIG. 2 shows microarrays of colon cancer cell lines using the Agilent platform.

FIG. 3 shows the calculated IC₅₀ for dasatinib on colon cancer cell lines.

FIG. 4 shows levels of polynucleotide and/or polypeptide expression in the dasatinib-sensitive cell lines as compared to the dasatinib-insensitive cell lines.

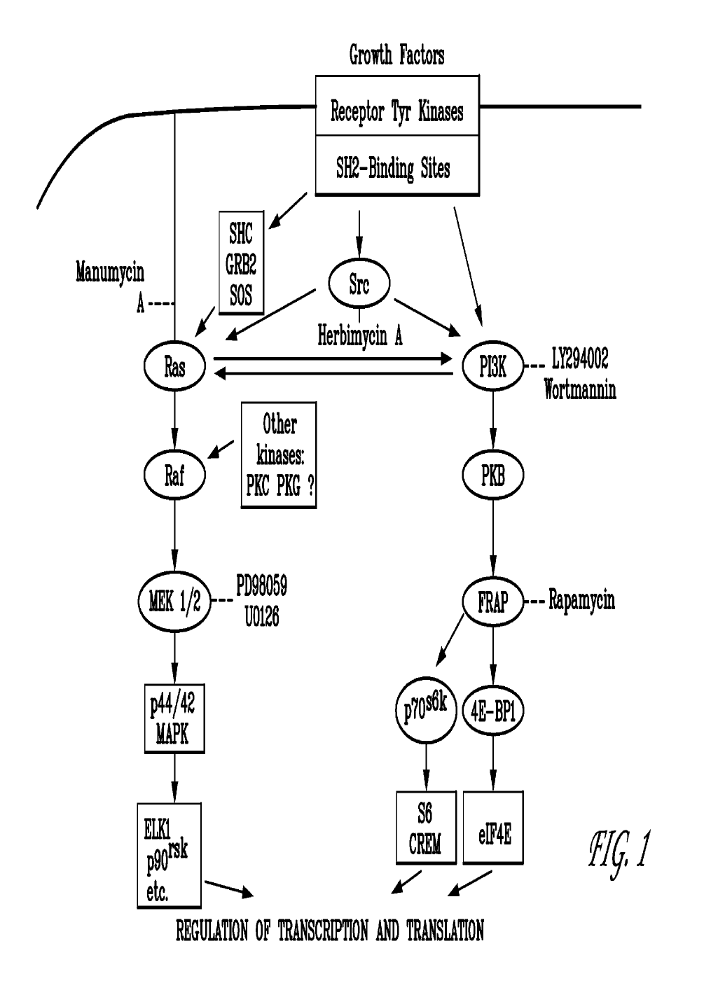

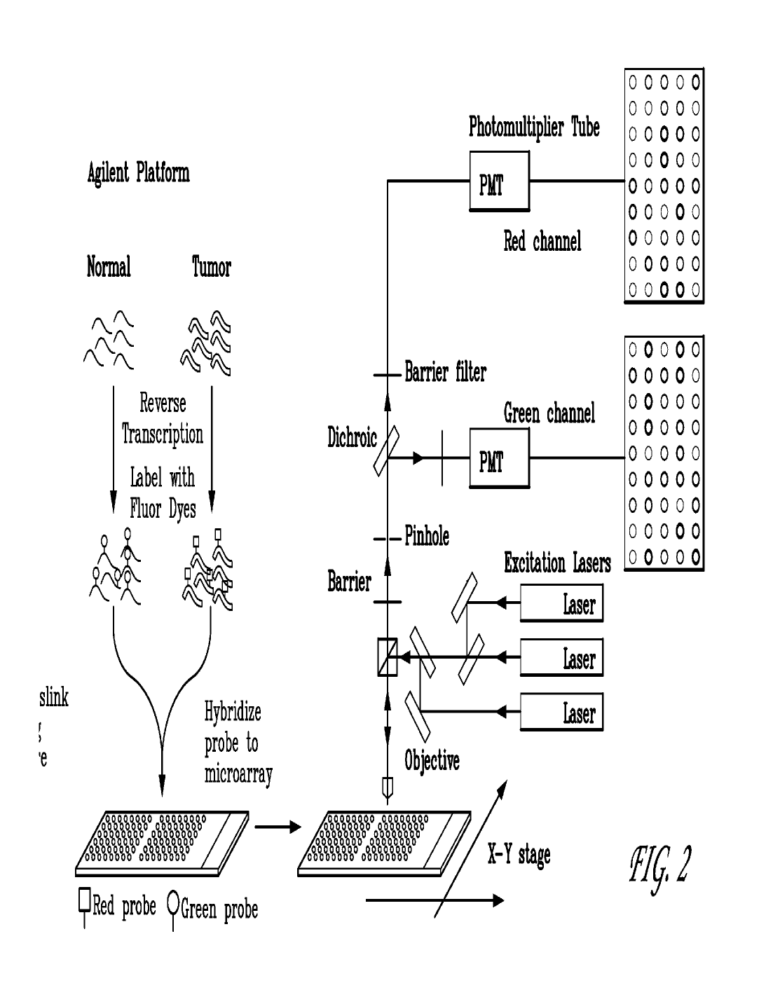

### DETAILED DESCRIPTION

The present invention relates to methods of using certain polynucleotides and/or polypeptides that are differentially expressed in colon cancer subtypes to predict sensitivity or resistance to therapy with abl kinase inhibitors and src kinase inhibitors, collectively protein tyrosine kinase inhibitors. In particular, the present invention relates to the discovery that PTK-7, PLK-2, and PLK-3 polynucleotides and/or polypeptides are differentially expressed in colon cancer subtypes that are sensitive to therapy with N-(2-chloro-6-methylphenyl)-2-(6-(4-(2-hydroxyethyl)piperazin-1-yl)-2-methylpyrimidin-4-ylamino)thiazole-5-carboxamide. There is a great need for new effective agents to treat patients with colon cancer. Currently there is interest in developing compounds that target specific molecular alterations in cancer cells to lead to better outcomes. However, critical to the development of these agents is the identification of patients most likely to respond to any given agent. Dasatinib is a small molecule tyrosine kinase inhibitor of the ABL and Src kinases. Using laboratory models of human colon cancer, the present inventors determined the in vitro sensitivity of these models to dasatinib. The present inventors observed that some of the cell lines were more sensitive to the compound than others in the laboratory. The present inventors then analyzed whole genome expression data on these cell lines to identify expressed polynucleotides and/or polypeptides that would allow us to differentiate colon cancers that would be more likely to respond to dasatinib than others. The present inventors successfully identified a group of polynucleotides and/or polypeptides whose expression is higher in the cell lines that are sensitive to dasatinib than in those that are resistant. The significance of this observation is that these polynucleotides and/or polypeptides could possibly identify patients that are more likely to respond to dasatinib in the clinic. If validated in a clinical trial, then these polynucleotides and/or polypeptides may help us identify which patients should receive dasatinib. In addition, these polynucleotides and/or polypeptides may be associated with response to other drugs that are in the same family as dasatinib. Further, if these polynucleotides and/or polypeptides are associated with response, there would likely be interest in developing a diagnostic assay for them. Cell lines that are “resistant” to dasatinib have a phenotype that encompasses either resistant, tolerant, unresponsive, or less susceptible to the growth inhibitory activity of dasatinib.

The purpose of this work was to identify a molecular subgroup of human colon cancer patients that would be more likely to respond to the multi-kinase Src and ABL inhibitor dasatinib. The experiments described herein identified that in a panel of 27 human colon cancer cell lines only a subset are sensitive to inhibition by dasatinib. Using global gene expression profiles the present inventors had previously generated using the Agilent platform, the present inventors queried what differentially expressed polynucleotides and/or polypeptides would identify these cell lines. Our analysis identified a subset of polynucleotides and/or polypeptides whose expression was higher in those cell lines sensitive to dasatinib. These polynucleotides and/or polypeptides may have clinical utility in predicting which patients in the clinic would be more likely to benefit from dasatinib.

Elevated levels of Src kinase have been implicated in the malignant potential of colorectal cancer. Dasatinib is an orally active, multi-targeted tyrosine kinase inhibitor of ABL and Src family kinases and is currently being investigated in solid tumors. Pre-clinical studies have identified potential biomarkers associated with response to dasatinib in breast cancer in vitro. To better understand the potential role of dasatinib in colon cancer, the present inventors used a panel of 27 human colon cancer cell lines which have undergone microarray analysis using the Agilent platform to determine their baseline expression. Using in vitro proliferation assays, the present inventors generated dose response curves and calculated IC50 values. Cell lines were then classified as sensitive and resistant using a cut-off of 100 nM. The present inventors then analyzed the microarray data to identify potential polynucleotides and/or polypeptides that are associated with response in vitro. Data matrices were used to select differentially expressed polynucleotides and/or polypeptides that would discriminate between sensitive and resistant cell lines. The data was analyzed using Rosetta Resolver software using both hierarchical and non-hierarchical methods. The present inventors identified a 3 marker-set that was able to distinguish sensitive and resistant cell lines. These 3 polynucleotides and/or polypeptides (PTK-7, PLK-2 and PLK-3) all had relatively higher levels of expression in the dasatinib sensitive cell lines as compared to the dasatinib-insensitive cell lines. Interestingly, two of these polynucleotides and/or polypeptides (PLK-2 and PLK-3) are part of the Polo-like kinase family, and have recently been identified as potential targets for cancer therapy themselves. In conclusion, these data (1) suggest a role for dasatinib in the treatment of human colorectal cancer (2) identify potential markers of response and (3) suggest rationale for combining dasatinib with PLK targeted agents. Clinical studies are required to validate these findings.

A “biomarker” or “marker” is an organic biomolecule which is differentially present in a sample taken from a subject of one phenotypic status (e.g., having a disease sensitive to dasatinib therapy) as compared with another phenotypic status (e.g., having a disease resistant to dasatinib therapy). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, can provide measures of relative risk that a subject belongs to one phenotypic status or another. Therefore, they are useful as markers for disease (diagnostics), therapeutic effectiveness of a drug (theranostics) and drug toxicity. The polynucleotide and polypeptides described herein can be used as biomarkers for certain cancers described herein. In particular, the present inventors have identified three polynucleotides and/or polypeptides, PTK-7, PLK-2, and PLK-3 that are differentially expressed in cancerous cells that are sensitive to therapy with N-(2-chloro-6-methylphenyl)-2-[[6-[4-(2-hydroxyethyl)-1-piperazinyl]-2-methyl-4-pyrimidinyl]amino]-5-thiazolecarboxamide.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Ausubel et al., eds., 1995, Current Protocols in Molecular Biology, Wiley and Sons. As appropriate, procedures involving the use of commercially available kits and reagents are generally carried out in accordance with manufacturer defined protocols and/or parameters unless otherwise noted.

It is to be understood that this invention is not limited to particular methods, reagents, compounds, compositions, or biological systems, which can, of course, vary. It is also to be understood that the terminology used herein is for the purpose of describing particular aspects only, and is not intended to be limiting.

As used in this specification and the appended claims, the singular forms “a”, “an”, and “the” include plural referents unless the content clearly dictates otherwise. Thus, for example, reference to “a peptide” includes a combination of two or more peptides, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Polynucleotide” refers to a polymeric form of nucleotides of at least about 10 bases or base pairs in length, either ribonucleotides or deoxynucleotides or a modified form of either type of nucleotide, and is meant to include single and double stranded forms of DNA.

“Polypeptide,” “peptide” and “protein” are used interchangeably herein to refer to a polymer of a polymer of at least about 6 amino acids. Throughout the specification, standard three letter or single letter designations for amino acids are used. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The phrase “specifically binds to” refers to a binding reaction which is determinative of the presence of a target in the presence of a heterogeneous population of other biologics. Thus, under designated assay conditions, the specified binding region bind preferentially to a particular target and do not bind in a significant amount to other components present in a test sample. Specific binding to a target under such conditions can require a binding moiety that is selected for its specificity for a particular target. A variety of assay formats can be used to select binding regions that are specifically reactive with a particular analyte. Typically a specific or selective reaction will be at least twice background signal or noise and more typically more than 10 times background. For purposes of the present invention, compounds, for example small molecules, can be considered for their ability to specifically bind to mutants described herein.

“Cancer” refers to any of a number of diseases that are characterized by uncontrolled, abnomial proliferation of cells, the ability of affected cells to spread locally or through the bloodstream and lymphatic system to other parts of the body (i.e., metastasize) as well as any of a number of characteristic structural and/or molecular features. The term cancer includes, but is not limited to, cancers of the gastrointestinal system including, but not limited to, stomach cancer, esophageal cancer, small bowel cancer or colon cancer; lung cancer; breast cancer; renal cell carcinoma; ovarian cancer, cervical cancer and uterine cancer; Hodgkin's lymphoma; Non-Hodgkin's lymphoma; cancers of the genitourinary system including, but not limited to, kidney cancer, prostate cancer, bladder cancer, and urethral cancer; cancers of the head and neck; liver cancer; c; cancers of the biliary tree; pancreatic cancer; cancers of the male reproductive system including, but not limited to, testicular cancer; Gestational trophoblastic disease; cancers of the endocrine system including, but not limited to, thyroid cancer, parathyroid cancer, adrenal gland cancer, carcinoid tumors, insulinomas and PNET tumors; sarcomas, including but not limited to, Ewing's sarcoma, osteosarcoma, liposarcoma, leiomyosarcoma, and rhabdomyosarcoma; mesotheliomas; cancers of the skin; melanomas; cancers of the central nervous system; pediatric cancers; and cancers of the hematopoietic system including, but not limited to all forms of leukemia, myelodysplastic syndromes, myeloproliferative disorders and multiple myeloma.

Additional examples of cancers include, for example, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia (AML), and chronic lymphocytic leukemia (CML).

“Leukemia” refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood—leukemic or aleukemic (subleukemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia. In certain aspects, the present invention provides treatment for chronic myeloid leukemia, acute lymphoblastic leukemia, and/or Philadelphia chromosome positive acute lymphoblastic leukemia (Ph+ALL).

“Hybridize”, “hybridizing”, “hybridizes” and the like, used in the context of polynucleotides, refers to conventional hybridization conditions, preferably such as hybridization in 50% formamide/6×SSC/0.1% SDS/100 μg/ml ssDNA, in which temperatures for hybridization are above 37° C. and temperatures for washing in 0.1×SSC/0.1% SDS are above 55° C., and most preferably to stringent hybridization conditions.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent condition” or “high stringency conditions” can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C.

“Moderately stringent conditions” can be identified as described by Sambrook et al., 1989, Molecular Cloning: A Laboratory Manual, New York: Cold Spring Harbor Press, and include the use of washing solution and hybridization conditions (e.g., temperature, ionic strength and %SDS) less stringent than those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

“Substantial identity” to a specified sequence refers to 80% identity or greater, i.e., 81%, 82%, 83%, 84%, 85%, 86%, 87%, 88%, 89%, 90%, 91%, 91%, 93%, 94%, 95%, 96%, 97%, 98%, 99%, 99.5%, 99.9%, or 100% identity to the specified sequence.

In the context of amino acid sequence comparisons, “identity” is used to express the percentage of amino acid residues at the same relative positions that are the same. Also in this context, “homology” is used to express the percentage of amino acid residues at the same relative positions that are either identical or are similar, using the conserved amino acid criteria of BLAST analysis, as is generally understood in the art. For example, % identity values can be generated by WU-BLAST-2 (Altschul et al., 1996, Methods in Enzymology 266:460-480; blast.wustl/edu/blast/README.html). Further details regarding amino acid substitutions, which are considered conservative under such criteria, are provided below.

“cDNAs” refers to complementary DNA that are mRNA molecules present in a cell or organism made into cDNA with an enzyme such as reverse transcriptase. A “cDNA library” is a collection of all of the mRNA molecules present in a cell or organism, all turned into cDNA molecules with the enzyme reverse transcriptase or an equivalent, then inserted into “vectors” (other DNA molecules that can continue to replicate after addition of foreign DNA). Exemplary vectors for libraries include bacteriophage (also known as “phage”), viruses that infect bacteria, for example, lambda phage. The library can then be probed for the specific cDNA (and thus mRNA) of interest.

“Expression” refers to the process by which polynucleotides are transcribed into mRNA and/or the process by which the transcribed mRNA is subsequently being translated into polypeptides. If the polynucleotide is derived from genomic DNA, expression may include splicing of the mRNA in a eukaryotic cell. “Differentially expressed” as applied to a polynucleotide and/or polypeptide, refers to the differential production of the mRNA transcribed and/or translated from the gene or the protein product encoded by the gene. A differentially expressed gene can be overexpressed or underexpressed as compared to the expression level of a normal or control cell. In one aspect, it refers to a differential that is 1.2, 1.3, 1.4, 1.5 times, 2 times, 2.5 times, 3 times, 4 times, 5 times, or even 10 times higher or lower than the expression level detected in a control sample. The term “differentially expressed” also refers to nucleotide sequences in a cell or tissue which are expressed where silent in a control cell or not expressed where expressed in a control cell. The level of expression of a PTK-7, PLK-2, and/or PLK-3 polynucleotide and/or polypeptide can be determined, for example, by determining the level of PTK-7, PLK-2, and/or PLK-3 polynucleotides or polypeptides.

“Determining the level” or “detecting the level” or “evaluating the level” means detecting the presence or absence of an analyte in a sample or quantifying the amount in relative or absolute terms. A relative amount could be, for example, high, medium or low. An absolute amount could reflect the measured strength of a signal or the translation of this signal strength into another quantitative format, such as micrograms/ml.

Additional definitions are provided throughout the subsections that follow.

### Exemplary Indications, Conditions, Diseases, and Disorders

The present invention provides methods of determining responsiveness of an individual having cancer, such as colon cancer, a protein tyrosine kinase-associated disorder, or a BCR-ABL associated disorder, to a certain treatment regimen and methods of treating an individual having a BCR-ABL associated disorder.

The term “BCR-ABL” as used herein is inclusive of both wild-type and mutant BCR-ABL.

“BCR-ABL associated disorders” are those disorders which result from BCR-ABL activity, including mutant BCR-ABL activity, and/or which are alleviated by the inhibition of BCR-ABL, including mutant BCR-ABL, expression and/or activity. A reciprocal translocation between chromosomes 9 and 22 produces the oncogenic BCR-ABL fusion protein. The phrase “BCR-ABL associated disorders” is inclusive of “mutant BCR-ABL associated disorders”.

Disorders included in the scope of the present invention include, for example, colon cancer, breast cancer, prostate cancer, leukemias, including, for example, chronic myeloid leukemia, acute lymphoblastic leukemia, and Philadelphia chromosome positive acute lymphoblastic leukemia (Ph+ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia, chronic lymphocytic leukemia, mastocytosis and any symptom associated with mastocytosis. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, and mast cell leukemia. Various additional cancers are also included within the scope of protein tyrosine kinase-associated disorders including, for example, the following: carcinoma, including that of the bladder, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma. In certain preferred embodiments, the disorder is leukemia, breast cancer, prostate cancer, lung cancer, colon cancer, melanoma, or solid tumors. In certain preferred embodiments, the leukemia is chronic myeloid leukemia (CML), Ph+ALL, AML, imatinib-resistant CML, imatinib-intolerant CML, accelerated CML, lymphoid blast phase CML.

A “solid tumor” includes, for example, sarcoma, melanoma, carcinoma, prostate carcinoma, lung carcinoma, colon carcinoma, breast carcinoma, or other solid tumor cancer.

“Mutant BCR-ABL associated disorder” is used to describe a BCR-ABL associated disorder in which the cells involved in said disorder are or become resistant to treatment with a kinase inhibitor used to treat said disorder as a result of a mutation in BCR-ABL. For example, a kinase inhibitor compound can be used to treat a cancerous condition, which compound inhibits the activity of wild type BCR-ABL which will inhibit proliferation and/or induce apoptosis of cancerous cells. Over time, a mutation can be introduced into the gene encoding BCR-ABL kinase, which can alter the amino acid sequence of the BCR-ABL kinase and cause the cancer cells to become resistant, or at least partially resistant, to treatment with the compound. Alternatively, a mutation can already be present within the gene encoding BCR-ABL kinase, either genetically or as a consequence of an oncogenic event, independent of treatment with a protein tyrosine kinase inhibitor, which can be one factor resulting in these cells propensity to differentiate into a cancerous or proliferative state, and also result in these cells being less sensitive to treatment with a protein tyrosine kinase inhibitor. Such situations are expected to result, either directly or indirectly, in a “mutant BCR-ABL kinase associated disorder” and treatment of such condition will require a compound that is at least partially effective against the mutant BCR-ABL, preferably against both wild type BCR-ABL and the mutant BCR-ABL. In the instance where an individual develops at least partial resistance to the kinase inhibitor imatinib, the mutant BCR-ABL associated disorder is one that results from an imatinib-resistant BCR-ABL mutation, or a protein tyrosine kinase inhibitor resistant BCR-ABL mutation. Similarly, in the instance where an individual develops at least partial resistance to the kinase inhibitor N-(2-chloro-6-methylphenyl)-2-[[6-[4-(2-hydroxyethyl)-1-piperazinyl]-2-methyl-4-pyrimidinyl]amino]-5-thiazolecarboxamide, the mutant BCR-ABL associated disorder is one that results from an N-(2-chloro-6-methylphenyl)-2-[[6-[4-(2-hydroxyethyl)-1-piperazinyl]-2-methyl-4-pyrimidinyl]amino]-5-thiazolecarboxamide resistant BCR-ABL mutation, or a protein tyrosine kinase inhibitor resistant BCR-ABL mutation.

Protein tyrosine kinase-associated disorders of particular interest herein are those disorders which result, at least in part, from aberrant SRC or BCR-ABL (WT or mutant) activity and/or which are alleviated by the inhibition of SRC or BCR-ABL (WT or mutant) referred to herein as “SRC associated disorders”, “SRC associated cancer”, or “BCR-ABL associated disorders”, “BCR-ABL associated cancer”

“SRC”, “SRC kinase”, and “Mutant SRC kinase” encompasses a SRC kinase with an amino acid sequence that differs from wild type SRC kinase by one or more amino acid substitutions, additions or deletions, and necessarily includes BCR-ABL encoding polynucleotides and polypeptides with one or more amino acid substitutions, additions, or deletions. SRC necessarily encompasses ABL, BCR/ABL, SRC including SRC family kinases such as c-Src, SRC/ABL, and other forms including, but not limited to, JAK, FAK, FPS, CSK, SYK, and BTK.

“N-(2-chloro-6-methylphenyl)-2-[[6-[4-(2-hydroxyethyl)-1 -piperazinyl]-2-methyl-4-pyrimidinyl]amino]-5-thiazolecarboxamide-resistant BCR-ABL mutation” refers to a specific mutation in the amino acid sequence of BCR-ABL that confers upon cells that express said mutation at least partial resistance to treatment with N-(2-chloro-6-methylphenyl)-2-[[6-[4-(2-hydroxyethyl)-1-piperazinyl]-2-methyl-4-pyrimidinyl]amino]-5-thiazolecarboxamide.

“Imatinib-resistant CML” refers to a CML in which the cells involved in CML are resistant to treatment with imatinib. Generally it is a result of a mutation in BCR-ABL.

“Imatinib-intolerant CML” refers to a CML in which the individual having the CML is intolerant to treatment with imatinib, i.e., the toxic and/or detrimental side effects of imatinib outweigh any therapeutically beneficial effects.

The invention provides a method of treating cancers, including both primary and metastatic cancers, including solid tumors such as those of the colon, breast, and prostate, as well as lymphomas and leukemias (including CML, AML and ALL), cancers of endothelial tissues, and including cancers which are resistant to other therapies, including other therapies involving administration of kinase inhibitors such as imatinib. Specifically, and without limitation, the invention provides the use of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib; a combination of N-(2-chloro-6-methylphenyl)-2-[[6-[4-(2-hydroxyethyl)-1-piperazinyl]-2-methyl-4-pyrimidinyl]amino]-5-thiazolecarboxamide, either alone or in combination with a tubulin stabilizing agent (e.g., pacitaxol, epothilone, taxane, etc.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a famysyl transferase inhibitor (e.g., (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile, hydrochloride salt); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor; an increased dosing frequency regimen of N-(2-chloro-6-methylphenyl)-2-[[6-[4-(2-hydroxyethyl)-1-piperazinyl]-2-methyl-4-pyrimidinyl]amino]-5-thiazolecarboxamide; and any other combination or dosing regimen comprising N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein, for treating disorders, for example cancers, which are resistant to other therapies involving administration of kinase inhibitors such as imatinib.

### Treatment Regimens

The invention encompasses treatment methods based upon the demonstration that patients harboring different levels of expression of the PTK-7, PLK-2, and/or PLK-3 polynucleotide and/or polypeptide have varying degrees of resistance and/or sensitivity to therapy with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof. Thus the methods of the present invention can be used, for example, in determining whether or not to treat an individual with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof; whether or not to treat an individual with a more aggressive dosage regimen of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof; or whether or not to treat an individual with combination therapy, i.e., a combination of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof with an additional anti-cancer therapy. The terms “treating”, “treatment” and “therapy” as used herein refer to curative therapy, prophylactic therapy, preventative therapy, and mitigating disease therapy.

For use herein, a BCR-ABL inhibitor refers to any molecule or compound that can at least partially inhibit BCR-ABL or mutant BCR-ABL activity or expression. These include inhibitors of the Src family kinases such as BCR/ABL, ABL, c-Src, SRC/ABL, and other forms including, but not limited to, JAK, FAK, FPS, CSK, SYK, and BTK. A series of inhibitors, based on the 2-phenylaminopyrimidine class of pharmacophotes, has been identified that have exceptionally high affinity and specificity for Abl (see, e.g., Zimmerman et al., Bioorg, Med. Chem. Lett. 7, 187 (1997)). All of these inhibitors are encompassed within the term a BCR-ABL inhibitor. Imatinib, one of these inhibitors, also known as STI-571 (formerly referred to as Novartis test compound CGP 57148 and also known as Gleevec), has been successfully tested in clinical trial a therapeutic agent for CML. AMN107, is another BCR-ABL kinase inhibitor that was designed to fit into the ATP-binding site of the BCR-ABL protein with higher affinity than imatinib. In addition to being more potent than imatinib (IC50<30 nM) against wild-type BCR-ABL, AMN107 is also significantly active against 32/33 imatinib-resistant BCR-ABL mutants. In preclinical studies, AMN107 demonstrated activity in vitro and in vivo against wild-type and imatinib-resistant BCR-ABL-expressing cells. In phase I/II clinical trials, AMN107 has produced haematological and cytogenetic responses in CML patients, who either did not initially respond to imatinib or developed imatinib resistance (Weisberg et al., British Journal of Cancer (2006) 94, 1765-1769, incorporated herein by reference in its entirety and for all purposes). SKI-606, NS-187, AZD0530, PD180970, CGP76030, and AP23464 are all examples of kinase inhibitors that can be used in the present invention. SKI-606 is a 4-anilino-3-quinolinecarbonitrile inhibitor of Abl that has demonstrated potent antiproliferative activity against CML cell (Golas et al., Cancer Research (2003) 63, 375-381). AZD0530is a dual Abl/Src kinase inhibitor that is in ongoing clinical trials for the treatment of solid tumors and leukemia (Green et al., Preclinical Activity of AZD0530, a novel, oral, potent, and selective inhibitor of the Src family kinases. Poster 3161 presented at the EORTC-NCI-AACR, Geneva Switzerland 28 Sep. 2004). PD180970 is a pyrido[2,3-d]pyrimidine derivative that has been shown to inhibit BCR-ABL and induce apoptosis in BCR-ABL expressing leukemic cells (Rosee et al., Cancer Research (2002) 62, 7149-7153). CGP76030 is dual-specific Src and Abl kinase inhibitor shown to inhibit the growth and survival of cells expressing imatinib-resistant BCR-ABL kinases (Warmuth et al., Blood, (2003) 101(2), 664-672). AP23464 is an ATP-based kinase inhibitor that has been shown to inhibit imatinib-resistant BCR-ABL mutants (O'Hare et al., Clin Cancer Res (2005) 11(19), 6987-6993). NS-187 is a selective dual Bcr-Abl/Lyn tyrosine kinase inhibitor that has been shown to inhibit imatinib-resistant BCR-ABL mutants (Kimura et al., Blood, 106(12):3948-3954 (2005)).

A “farnysyl transferase inhibitor” can be any compound or molecule that inhibits farnysyl transferase. The farnysyl transferase inhibitor can have formula (II), (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile, hydrochloride salt. The compound of formula (II) is a cytotoxic FT inhibitor which is known to kill non-proliferating cancer cells preferentially. The compound of formula (II) can further be useful in killing stem cells.

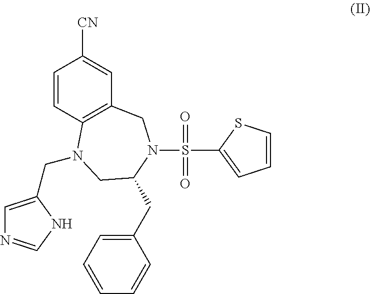

The compound of formula (II), its preparation, and uses thereof are described in U.S. Pat. No. 6,011,029, which is herein incorporated by reference in its entirety and for all purposes. Uses of the compound of formula (II) are also described in WO2004/015130, published Feb. 19, 2004, which is herein incorporated by reference in its entirety and for all purposes.

For use herein, combination therapy refers to the administration of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof with a second therapy at such time that both the second therapy and N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof, will have a therapeutic effect. Such administration can involve concurrent (i.e., at the same time), prior, or subsequent administration of the second therapy with respect to the administration of N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof.

Treatment regimens can be established based upon the presence of an elevated expression of one or more of PTK-7, PLK-2, and/or PLK-3, as disclosed herein. For example, the invention encompasses screening cells from an individual who may suffer from, or is suffering from, a disorder that is commonly treated with a kinase inhibitor, such as a protein tyrosine kinase inhibitor. Such a disorder can include, for example, colon cancer, or other cancers described herein. The level of expression of one or more cells from an individual, is determined using methods known in the art.

The actual dosage employed of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof can be varied depending upon the requirements of the patient and the severity of the condition being treated. Determination of the proper dosage for a particular situation is within the skill of the art. The effective amount of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof can be determined by one of ordinary skill in the art, and includes, exemplary dosage amounts for an adult human of from about 0.05 to about 100 mg/kg of body weight of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof, per day, which can be administered in a single dose or in the form of individual divided doses, such as from 1, 2, 3, or 4 times per day. It will be understood that the specific dose level and frequency of dosing for any particular subject can be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition.

A treatment regimen is a course of therapy administered to an individual suffering from a disease described herein that can include treatment with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof and/or other anti-cancer therapies. When more than one therapy is administered, the therapies can be administered concurrently or consecutively. Administration of more than one therapy can be at different times (i.e., consecutively) and still be part of the same treatment regimen.

Accordingly, in one aspect of the invention, if at least one member, at least two members (in any combination), or all three members of the PTK-7, PLK-2, and/or PLK-3 polynucleotide or polypeptide set are expressed in colon cancer cells as outlined herein, the treatment regimen may only require administration of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof to either kill or inhibit the proliferation of said cancer. Such administration may include a pharmaceutically acceptable amount of N-(2-chloro-6-methylphenyl)-2-[[6-[4-(2-hydroxyethyl)-1-piperazinyl]-2-methyl-4-pyrimidinyl]amino]-5-thiazolecarboxamide or a salt, hydrate, or solvate thereof, a combination of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof.

Alternatively, if at least one member, at least two members, or all three members of the PTK-7, PLK-2, and/or PLK-3 polynucleotide or polypeptide set are not expressed in colon cancer cells, or if less then optimal levels (e.g., levels that are high enough to predict sensitivity to dasatinib) of a PTK-7, PLK-2, and/or PLK-3 predictor polynucleotide set member expression is observed, the treatment regimen may require either increased dosing frequency or a higher dose of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof, and/or a combination of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof with another pharmaceutically acceptable agent including another anti-cancer agent such as a kinase inhibitor drug such as imatinib, AMN107, PD180970, GGP76030, AP23464, SKI 606, and/or AZD0530; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, and the like.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor (including (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile, hydrochloride salt described in U.S. Pat. No. 6,011,029); and any other combination or dosing regimen comprising N-(2-chloro-6-methylphenyl)-2-[[6-[4-(2-hydroxyethyl)-1-piperazinyl]-2-methyl-4-pyrimidinyl]amino]-5-thiazolecarboxamide. In one aspect, an increased dose of N-(2-chloro-6-methylphenyl)-2-[[6-[4-(2-hydroxyethyl)-1-piperazinyl]-2-methyl-4-pyrimidinyl]amino]-5-thiazolecarboxamide would be about 10, 20, 30, 40, 50, 60, 70, 80, 90, or 95% more than the typical N-(2-chloro-6-methylphenyl)-2-[[6-[4-(2-hydroxyethyl)-1-piperazinyl]-2-methyl-4-pyrimidinyl]amino]-5-thiazolecarboxamide dose for a particular indication or for individual, or about 1.5×, 2×, 2.5×, 3×, 3.5×, 4×, 4.5×, 5×, 6×, 7×, 8×, 9×, or 10× more N-(2-chloro-6-methylphenyl)-2-[[6-[4-(2-hydroxyethyl)-1-piperazinyl]-2-methyl-4-pyrimidinyl]amino]-5-thiazolecarboxamide than the typical N-(2-chloro-6-methylphenyl)-2-[[6-[4-(2-hydroxyethyl)-1-piperazinyl]-2-methyl-4-pyrimidinyl]amino]-5-thiazolecarboxamide dose for a particular indication or for individual. In particular, N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof may typically be administered 2 times per day at 70 mg, however, it can be dosed at, other amounts, for example, 50, 70, 90, 100, 110, or 120 BID, or 100, 140, or 180 once daily, twice daily, or thrice daily.

It will be understood that the specific dose level and frequency of dosing for any particular subject can be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats, and the like, subject to protein tyrosine kinase-associated disorders. The same also applies to any combination disclosed herein.

In practicing the many aspects of the invention herein, biological samples can be selected from many sources such as tissue biopsy (including cell sample or cells cultured therefrom; biopsy of solid tissue, for example cells from a solid tumor), blood, blood cells (red blood cells or white blood cells), serum, plasma, lymph, ascetic fluid, cystic fluid, urine, sputum, stool, saliva, bronchial aspirate, CSF or hair. Cells from a sample can be used, or a lysate of a cell sample can be used. In certain embodiments, the biological sample is a tissue biopsy cell sample or cells cultured therefrom, for example, cells removed from a solid tumor or a lysate of the cell sample. In certain embodiments, the biological sample comprises blood cells.

### Biomarkers and Biomarker Sets

The invention includes individual biomarkers and biomarker sets having both diagnostic and prognostic value in proliferative disease areas in which protein tyrosine kinase status is of importance, e.g., in cancers or tumors, or in disease states in which cell signaling and/or cellular proliferation controls are abnormal or aberrant. The biomarker sets comprise a plurality of biomarkers that highly correlate with resistance or sensitivity to one or more protein tyrosine kinase inhibitors.

The biomarkers and biomarker sets of the invention enable one to predict or reasonably foretell the likely effect of one or more protein tyrosine kinase inhibitors in different biological systems or for cellular responses merely based upon whether one or more of the biomarkers of the present invention are overexpressed relative to normal. The biomarkers and biomarker sets can be used in in vitro assays of cellular proliferation by sample cells to predict in vivo outcome. In accordance with the invention, the various biomarkers and biomarker sets described herein, or the combination of these biomarker sets with other biomarkers or markers, can be used, for example, to predict and monitor how patients with cancer might respond to therapeutic intervention with one or more protein tyrosine kinase inhibitors.

In specific embodiments of the present invention, overexpression of PTK-7, PLK-2, and/or PLK-3 correlated with response to protein tyrosine kinase inhibitors.

Measuring the level of expression of a biomarker and biomarker set provides a useful tool for screening one or more tumor samples before treatment of a patient with the protein tyrosine kinase inhibitors. The screening allows a prediction of whether the cells of a tumor sample will respond favorably to the protein tyrosine kinase inhibitors, based on the presence or absence of over-expression—such a prediction provides a reasoned assessment as to whether or not the tumor, and hence a patient harboring the tumor, will or will not respond to treatment with the protein tyrosine kinase inhibitors.

A difference in the level of the biomarker that is sufficient to indicate whether the mammal will or will not respond therapeutically to the method of treating cancer can be readily determined by one of skill in the art using known techniques. The increase or decrease in the level of the biomarker can be correlated to determine whether the difference is sufficient to identify a mammal that will respond therapeutically. The difference in the level of the biomarker that is sufficient can, in one aspect, be predetermined prior to determining whether the mammal will respond therapeutically to the treatment. In one aspect, the difference in the level of the biomarker is a difference in the mRNA level (measured, for example, by RT-PCR or a microarray), such as at least about a two-fold difference, at least about a three-fold difference, or at least about a four-fold difference in the level of expression, or more. In another aspect, the difference in the level of the biomarker is determined at the protein level by mass spectral methods or by FISH or by IHC. In another aspect, the difference in the level of the biomarker refers to a p-value of <0.05 in Anova analysis. In yet another aspect, the difference is determined in an ELISA assay.

The biomarker or biomarker set can also be used as described herein for monitoring the progress of disease treatment or therapy in those patients undergoing treatment for a disease involving a microtubulin-stabilizing agent.

The biomarkers also serve as targets for the development of therapies for disease treatment. Such targets may be particularly applicable to treatment of cancer, such as, for example, breast and/or lung cancer.

Indeed, because these biomarkers are differentially expressed in sensitive and resistant cells, their expression patterns are correlated with relative intrinsic sensitivity of cells to treatment with protein tyrosine kinase inhibitors. Accordingly, the biomarkers over expressed in resistant cells may serve as targets for the development of new therapies for the tumors which are resistant to protein tyrosine kinase inhibitors. The level of biomarker protein and/or mRNA can be determined using methods well known to those skilled in the art. For example, quantification of protein can be carried out using methods such as ELISA, 2-dimensional SDS PAGE, Western blot, immunoprecipitation, immunohistochemistry, fluorescence activated cell sorting (FACS), or flow cytometry. Quantification of mRNA can be carried out using methods such as PCR, array hybridization, Northern blot, in-situ hybridization, dot-blot, TAQMAN®, or RNAse protection assay.

The present invention encompasses the use of any one or more of the following as a biomarker for use in predicting response to a protein tyrosine kinase inhibitor: PTK-7, PLK-2, and/or PLK-3.

The present invention also encompasses any combination of the aforementioned biomarkers, including, but not limited to: PTK-7, PLK-2, and PLK-3; PTK-7 and PLK-3; PTK-7 and PLK-2; PLK-2 and PLK-3; PTK-71; PLK-2; and PLK-3.

Identification of biomarkers that provide rapid and accessible readouts of efficacy, drug exposure, or clinical response is increasingly important in the clinical development of drug candidates. Embodiments of the invention include measuring changes in the levels of mRNA and/or protein in a sample to determine whether said sample contains increased expression of PTK-7, PLK-2, and/or PLK-3. In one aspect, said samples serve as surrogate tissue for biomarker analysis. These biomarkers can be employed for predicting and monitoring response to one or more protein tyrosine kinase inhibitors. In one aspect, the biomarkers of the invention are one or more of the following: PTK-7, PLK-2, and/or PLK-3, including both polynucleotide and polypeptide sequences. In another aspect, the biomarkers of the invention are nucleotide sequences that, due to the degeneracy of the genetic code, encodes for a polypeptide sequence provided in the sequence listing.

The biomarkers serve as useful molecular tools for predicting and monitoring response to protein tyrosine kinase inhibitors.

Methods of measuring the level of any given marker described herein may be performed using methods well known in the art, which include, but are not limited to PCR; RT-PCR; FISH; IHC; immuno-detection methods; immunoprecipitation; Western Blots; ELISA; radioimmunoassays; PET imaging; HPLC; surface plasmon resonance, and optical spectroscopy; and mass spectrometry, among others.

The biomarkers of the invention may be quantified using any immunospecific binding method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds., Current Protocols in Molecular Biology, Vol. 1, John Wiley & Sons, Inc., New York (1994), which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% TRASYLOL®) supplemented with protein phosphatase and/or protease inhibitors (e.g., EDTA, PMSF, aprotinin, sodium vanadate), adding the antibody of interest (i.e., one directed to a biomarker of the present invention) to the cell lysate, incubating for a period of time (e.g., 1-4 hours) at 4° C., adding protein A and/or protein G SEPHAROSE® beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with SEPHAROSE® beads). For further discussion regarding immunoprecipitation protocols see, e.g., Ausubel et al., eds., Current Protocols in Molecular Biology, Vol. 1, John Wiley & Sons, Inc., New York at 10.16.1 (1994).

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds., Current Protocols in Molecular Biology, Vol. 1, John Wiley & Sons, Inc., New York at 10.8.1 (1994).

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds., Current Protocols in Molecular Biology, Vol. 1, John Wiley & Sons, Inc., New York at 11.2.1 (1994).

Alternatively, identifying the relative quantitation of the biomarker polypeptide(s) may be performed using tandem mass spectrometry; or single or multi dimensional high performance liquid chromatography coupled to tandem mass spectrometry. The method takes into account the fact that an increased number of fragments of an identified protein isolated using single or multi dimensional high performance liquid chromatography coupled to tandem mass spectrometry directly correlates with the level of the protein present in the sample. Such methods are well known to those skilled in the art and described in numerous publications, for example, 2-D Proteome Analysis Protocols, A. J. Link, ed., Humana Press (1999), ISBN: 0896035247; Mass Spectrometry of Proteins and Peptides, J. R. Chapman, ed., Humana Press (2000), ISBN: 089603609X.

As used herein the terms “modulate” or “modulates” or “modulators” refer to an increase or decrease in the amount, quality or effect of a particular activity, or the level of DNA, RNA, or protein detected in a sample.

### Kits

For use in the diagnostic and therapeutic applications described or suggested above, kits are also provided by the invention. Such kits can comprise a carrier means being compartmentalized to receive in close confinement one or more container means such as vials, tubes, and the like, each of the container means comprising one of the separate elements to be used in the method. For example, one of the container means can comprise a probe that is or can be detectably labeled. Such probe can be an antibody or polynucleotide specific for a PTK-7, PLK-2, and/or PLK-3 protein or polynucleotide, respectively. Where the kit utilizes nucleic acid hybridization to detect the target nucleic acid, the kit can also have containers containing nucleotide(s) for amplification of the target nucleic acid sequence and/or a container comprising a reporter-means, such as a biotin-binding protein, such as avidin or streptavidin, bound to a reporter molecule, such as an enzymatic, florescent, or radioisotope label.

A typical embodiment of the invention is a kit comprising a container, a label on said container, and a composition contained within said container; wherein the composition includes a PTK-7, PLK-2, and/or PLK-3 polynucleotide and/or polypeptide set specific antibody and/or a polynucleotide that hybridizes to a complement of the PTK-7, PLK-2, and/or PLK-3 polynucleotide under stringent conditions (or binds to a PTK-7, PLK-2, and/or PLK-3 polypeptide encoded by the polynucleotide). In certain aspects, the label on said container indicates that the composition can be used to evaluate the presence of PTK-7, PLK-2, and/or PLK-3 protein, RNA or DNA in at least one type of mammalian cell, and includes instructions for using the PTK-7, PLK-2, and/or PLK-3 antibody and/or polynucleotide for evaluating the presence of PTK-7, PLK-2, and/or PLK-3 protein, RNA or DNA in at least one type of mammalian cell.

The kit of the invention will typically comprise the container described above and one or more other containers comprising materials desirable from a commercial and user standpoint, including buffers, diluents, filters, needles, syringes, and package inserts with instructions for use. A label can be present on the container to indicate that the composition is used for a specific therapy or non-therapeutic application, and may also indicate directions for either in vivo or in vitro use, such as those described above.

Other embodiments and uses will be apparent to one skilled in the art in light of the present disclosures.

The invention will be further described with reference to the following examples; however, it is to be understood that the invention is not limited to such examples.

### Exemplary Aspects

### EXAMPLE 1

### Materials and Methods

Proliferation Assays. The effects of dasatinib on cell growth were studied in human colon cancer cell lines growing in vitro. Cells were seeded in duplicate at 5,000 to 10,000 cells per well in 24-well plates. The day after plating, dasatinib was added over six two-fold dilutions to generate a dose response curve. Control wells without drug were seeded as well. Both the adherent and floating fractions of cells were counted on day 6 for both treatment and control wells. After trypsinization cells were placed in Isotone solution and counted immediately using a Coulter Z2 particle counter (Beckman Coulter Inc, Fullerton, Calif.). Growth inhibition was calculated [(1-experimental value/control value)×100] adding both the values obtained for the floating cells and the adherent cells.

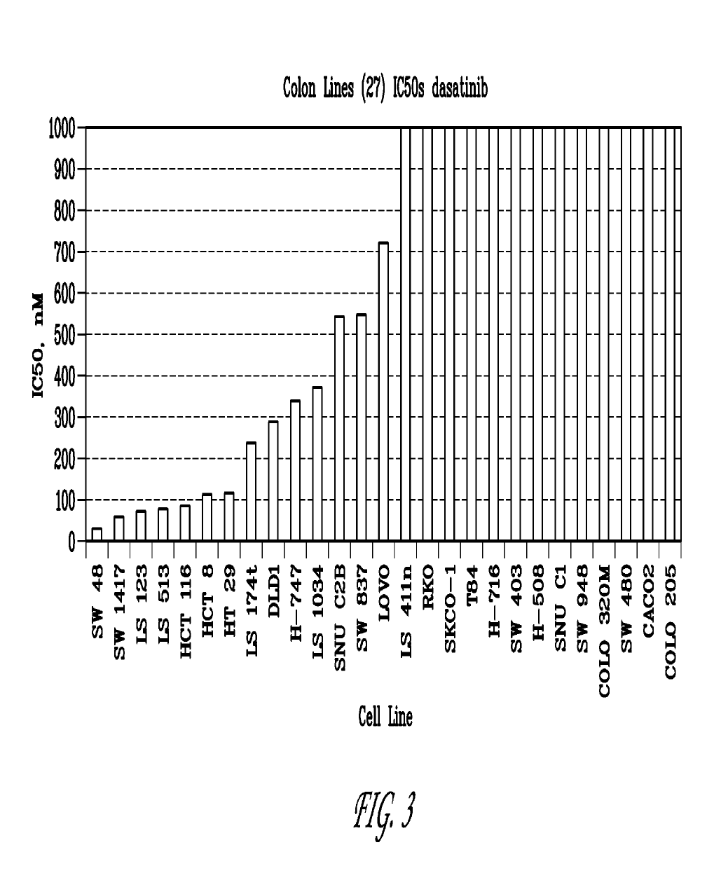

Microarray Analysis. Cells were grown to log phase and then RNA was extracted using the RNeasy Kit (Qiagen). Microarrays of colon cancer cell lines were then performed on the Agilent Human 1A V1 chip (FIG. 2). Microarray slides were read using an Agilent Scanner and the Agilent Feature Extraction software version 7.5 was used to calculate expression values. Cluster analysis was performed in Resolver and cell line profiles were exported to Excel (Microsoft) for additional analysis of the distribution of expression changes.

### EXAMPLE 2

### Analysis of Src by Microarray

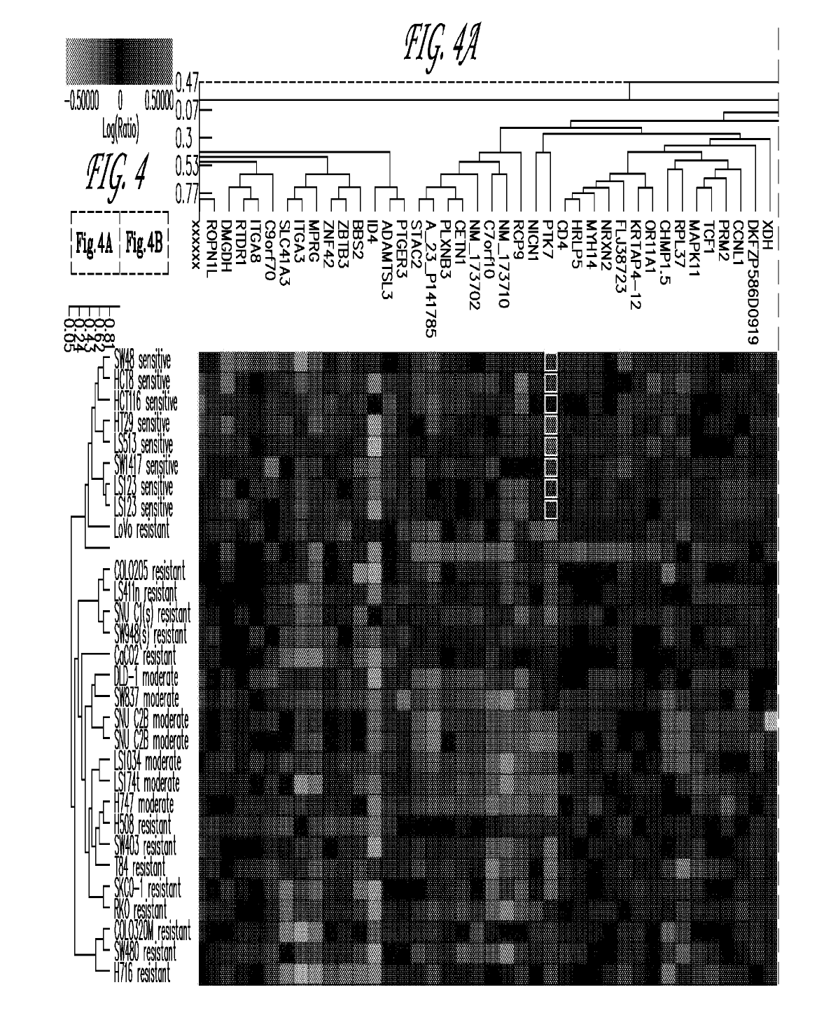

Based on the proposed role of Src in colorectal cancer, the present inventors assembled a panel of 27 human colon cancer cell lines and molecularly characterized them using the Agilent microarray platform. Using in vitro proliferation assays, the present inventors generated dose response curves and calculated IC50 values. There is a spectrum of sensitivity to dasatinib including a subset with IC50 values well below clinically achievable serum concentrations (<100 nM). In addition, most of the resistant cell lines had almost no growth inhibition even at very high doses of dasatinib (FIG. 3). Furthermore, Src expression was not associated with response and no Src mutations were identified in the kinase domain. The calculated IC50 values for other kinases is provided in the following table.

| kinase | enzyme IC₅₀, nM |
|---|---|
| Bcr-Abl | <1.0 |
| src | 0.50 |
| lck | 0.40 |
| yes | 0.50 |
| c-kit | 5.0 |
| PDGFRβ | 28 |
| p38 | 100 |
| Her1 | 180 |
| Her2 | 710 |
| FGFR-1 | 880 |
| MEK | 1700 |
| VEGFR-2 | >2000 |
| CDK2 | >5000 |
| IKK | >10000 |
| AKT | >50000 |
| FAK | >50000 |
| IGF-1R | >50000 |
| IR | >50000 |
| MK2 | >50000 |
| PKC α, δ, τ, ζ | >50000 |

### EXAMPLE 3

### Data Matrices to Select Differentially Expressed Polynucleotides and Polypeptides

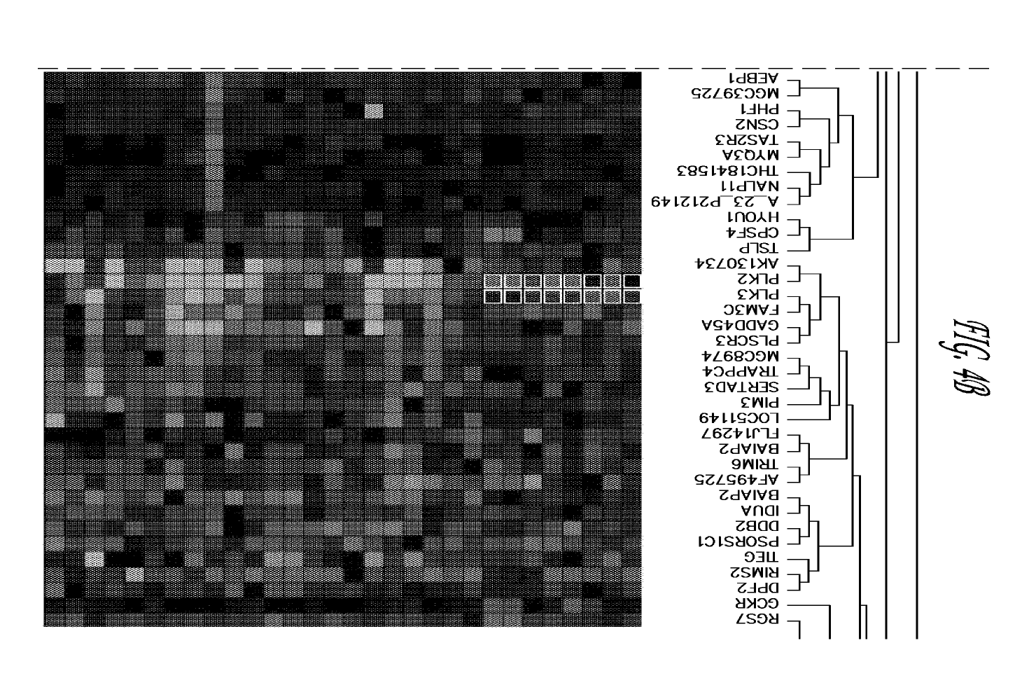

To identify potential polynucleotides and/or polypeptides that are associated with response to dasatinib in colon cancer in vitro, the present inventors developed data matrices to select differentially expressed polynucleotides and/or polypeptides that would discriminate between sensitive and resistant cell lines. The present inventors were able to identify a 3 polynucleotide and/or polypeptide-set that was able to distinguish between those cell lines that were resistant and sensitive to dasatinib. These 3 polynucleotides and/or polypeptides (PTK-7, PLK-2 and PLK-3) all had relatively higher levels of expression in the dasatinib-sensitive cell lines as compared to the dasatinib-insensitive cell lines (FIG. 4). Again, as the present inventors have seen with a number of targeted agents, measurement of the “target” itself (here Src) does not necessarily correlate with response.

### EXAMPLE 4

### Clinical Role for Dastinib in Colon Cancer

Using pre-clinical models of human colon cancer the present inventors have identified a sub-group of cell lines more sensitive to dasatinib in vitro.

The present inventors identified a group of differentially expressed polynucleotides and/or polypeptides that are expressed more highly in sensitive lines—interestingly Src is not one of the polynucleotides and/or polypeptides.

These data suggest a potential clinical role for dasatinib in colon cancer as a single agent, and in combination with other targeted agents (i.e. PLK inhibitors). Clinical studies are needed to validate these findings.

References of potential relevance to the subject matter disclosed herein include the following, which are incorporated herein by reference in their entirety for all purposes.

1. Flamigni F, Facchini F, Cappani C, et al. p44/42 mitogen-activated protein kinase is involved in the expression of ornithine decarboxylase in leukaemia L1210 cells. Biochem J; 341: 363-9, 1999.

2. Lombardo L J, Lee F Y, Chen P, et al. Discovery of N-(2-chloro-6-methyl-phneyl)-2-(6-(4-(2-hydroxyethyl)-piperazin-1-yl)-2-methylpyrimidin-4-ylamino)thiazole-5-carboxamide (BMS-354825), a dual Src/Abl kinase inhibitor with potent antitumor activity in pre-clinical assays. J Med Chem,: 47(27):6658-6661, 2004.

3. Finn R S, Dering J, Ginther C, et al. Dasatinib, an orally active small molecule inhibitor of both the src and abl kinases, selectively inhibits growth of basal-type/“triple-negative” breast cancer cell lines growing in vitro. Breast Cancer Res Treat. February 2007.

4. Huang F, Reeves K, Han X et al. Identification of candidate molecular markers predicting sensitivity in solid tumors to dasatinib: rationale for patient selection. Cancer Res. 67(5):2226-38, 2007.

5. Serrels A, Macpherson I R, Evans T R et al. Identification of potential biomarkers for measuring inhibition of Src kinase activity in colon cancer cells following treatment with dasatinib. Mol Cancer Ther. 5 (12) 3014-3022, 2006.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed:

1. A method for determining the responsiveness of a mammalian tumor cell to treatment with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof, the method comprising determining the level of expression of at least one marker selected from PTK-7, PLK-2, and PLK-3 in said tumor cell, wherein the level of expression is indicative of whether the cell is likely to respond or is responsive to the treatment.

2. The method of claim 1 wherein the level of expression of at least two markers selected from PTK-7, PLK-2, and PLK-3 in said tumor cell is determined.

3. The method of claim 1 wherein the level of expression is determined by detecting the level of mRNA transcribed from the at least one marker.

4. The method of claim 1 wherein the level of expression is determined by detecting the level of cDNA produced from the reverse transcription of the mRNA transcribed from the at least one marker.

5. The method of claim 1 wherein the level of expression is determined by detecting the level of the polypeptide encoded by the at least one marker.

6. A kit for use in characterizing a mammalian tumor cell comprising:
a means for determining the level of expression of at least one marker selected from PTK-7, PLK-2, and PLK-3 in said tumor cell, and
instructions for use and interpretation of the kit results.

7. The kit of claim 6 wherein the level of expression is determined by detecting the level of the polypeptide encoded by the at least one marker and the kit comprises at least one antibody that specifically binds to the polypeptide encoded by the at least one marker.

8. The kit of claim 6 wherein the level of expression is determined by detecting the level of mRNA transcribed from the at least one marker and the kit comprises at least one polynucleotide that hybridizes to the mRNA transcribed from the at least one marker.

9. A method for determining the responsiveness of an individual with cancer to treatment with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof, comprising:
obtaining a biological sample from said individual; and
determining the level of expression of at least one marker selected from PTK-7, PLK-2, and PLK-3 in the biological sample, wherein said level of expression is indicative of responsiveness to the treatment.

10. A method of treating an individual suffering from a cancer comprising:
determining the level of expression of at least one marker selected from PTK-7, PLK-2, and PLK-3 in a biological sample obtained from the subject; and
administering N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof to the individual if it the at least one marker is present in the biological sample at an increased level as compared to a control sample.

11. A method of evaluating tumor aggressiveness, comprising determining the level of expression of at least one marker selected from PTK-7, PLK-2, and PLK-3 in a tumor sample and correlating the level of expression to tumor aggressiveness.
